(19) United States
(12) Patent Application Publication
(10) Pub. No.: US 2005/0063921 A1
(43) Pub. Date: Mar. 24, 2005

(54) Oral composition

(75) Inventors: Dominique Charmot, Campbell, CA (US); Christopher David Gibbs, Bebington (GB); Oleg Kolosov, San Jose, CA (US); Mingjun Liu, Santa Clara, CA (US); Son Hoai Nguyen, Santa Clara, CA (US); Miroslav Petro, San Jose, CA (US); Steven Paul Rannard, Bebington (GB)

Correspondence Address: UNILEVER INTELLECTUAL PROPERTY GROUP, 700 SYLVAN AVENUE,, BLDG C2 SOUTH, ENGLEWOOD CLIFFS, NJ, 07632-3100, US

(73) Assignee: Unilever Home & Personal Care USA, Division of Conopco, Inc.

(21) Appl. No.: 10/666,489

(22) Filed: Sep. 19, 2003

(51) Int. Cl. A61K 9/68; A61K 7/16

(52) U.S. Cl. 424/48; 424/49

(57) ABSTRACT

Oral care composition comprising a polymer obtainable by copolymerising a mixture of comonomers, said mixture comprising: (a) a cationic monomer selected from (ar-vinylbenzyl) trimethylammonium chloride, (dimethylaminopropyl) methacrylamide, [2(methacryloyloxy)ethyl]trimethylammonium chloride, 2-aminoethylmethacrylate hydrochloride and mixtures thereof; and
(b) at least one anionic or neutral monomer selected from styrene, mono-2-(methacryloyl)ethyl succinate, vinyl acetate, N, N-dimethylacrylamide, 2-ethylhexylacrylate, vinylphosphonic acid, acrylic acid, 2-acrylamido-2-methyl-1-propanesulfonic acid, N-[tris(hydroxymethyl)methyl] acrylamide, N-vinylpyrrolidone, butyl acrylate, 2-hydroxyethylacrylate, polyethyleneglycol methylethermethacrylate and mixtures thereof, said oral care composition in the form of any one of a toothpaste, gel, foam, chewing gum, deformable strip or mouthwash and which is suitable for use in the oral cavity.

The present invention relates to an oral composition comprising a polymer which is delivered to the oral surfaces during toothbrushing.

We have found that there exists a range of polymers which are delivered more effectively to the oral surfaces during brushing. Accordingly, these polymers provide a useful tool for the delivery of active substances for the treatment or prevention of oral care related conditions such as gingivitis, caries, tartar, oral malodour, etc.

Accordingly the present invention provides an oral care composition comprising a polymer obtainable by copolymerising a mixture of comonomers, said mixture comprising:

(a) a cationic monomer selected from (ar-vinylbenzyl) trimethylammonium chloride, (dimethylaminopropyl) methacrylamide, [2(methacryloyloxy)ethyl]trimethylammonium chloride, 2-aminoethylmethacrylate hydrochloride and mixtures thereof; and

(b) at least one anionic or neutral monomer selected from styrene, mono-2-(methacryloyl)ethyl succinate, vinyl acetate, N,N-dimethylacrylamide, 2-ethylhexylacrylate, vinylphosphonic acid, acrylic acid, 2-acrylamido-2-methyl-1-propanesulfonic acid, N-[tris(hydroxymethyl)methyl] acrylamide, N-vinylpyrrolidone, butyl acrylate, 2-hydroxyethylacrylate, polyethyleneglycol methylethermethacrylate and mixtures thereof,

said oral care composition in the form of any one of a toothpaste, gel, foam, chewing gum, deformable strip or mouthwash and which is suitable for use in the oral cavity.

Preferred polymers include those polymers obtainable by copolymerising a mixture of (ar-vinylbenzyl) trimethylammonium chloride, styrene and a further neutral comonomer selected from N-[tris(hydroxymethyl)methyl] acrylamide and N-vinylprrolidone.

Further more preferred polymers include those polymers obtainable by copolymerising a mixture of (dimethylaminopropyl) methacrylamide with anionic and/or neutral comonomers selected from mono-2-(methacryloyl)ethyl succinate, vinyl acetate, butyl acrylate, N-[tris(hydroxymethyl)methyl] acrylamide and mixtures thereof.

Of these preferable polymers the most preferred polymers include the following mixtures of cationic comonomers and neutral and/or anionic comonomers:

(a) where the cationic comonomer is aminoethylmethacrylate hydrochloride and the neutral/anionic comonomer includes N, N-dimethylacrylamide, more preferably in a mol % ratio in the polymerisation mixture of from 25:75 to 95:5, more preferably from 50:50 to 90:10, most preferably of from 60:40 to 80:20. Especially preferred polymers of this comonomer combination type include those with a mol% ratio of around 75:25 in the copolymerisation mixture.

(b) where the cationic comonomer is (dimethylaminopropyl) methacrylamide and the neutral/anionic comonomer includes of the anionic comonomers mono-2-(methacryloyl)ethylsuccinate, 2-acryloamido-2-methyl-1-propanesulphonic acid and/or acrylic acid, and/or of the neutral hydrophilic comonomers N-[tris(hydroxymethyl)methyl]acrylamide and/or N, N-dimethylacrylamide, and/or of the neutral hydrophobic comonomers vinylacetate, butyl acrylate and/or 2-ethylhexylacrylate. The preferred further comonomer is either mono-2-(methacryloyl)ethylsuccinate or vinylacetate. Where the further comonomer is mono-2-(methacryloyl)ethylsuccinate it is preferred that a second further comonomer is present and that this is either N-[tris(hydroxymethyl)methyl]acrylamide or butyl acrylate. Where the further comonomer is vinylacetate it is preferred that it is used alone with the (dimethylaminopropyl) methacrylamide. More preferably the mol% ratio in the polymerisation mixture of this polymer type is from 10 to 90 N, N-dimethylacrylamide the remainder being the further comonomer(s).

(c) where the cationic comonomer is +82(methacryolyloxy)ethyl]trimethylammonium chloride and the neutral/anionic comonomer includes of the anionic comonomers mono-2-(methacryloyl)ethylsuccinate and/or acrylic acid and of the neutral hydrophilic comonomers 2-ethylhexylacrylate. Where the further comonomer is mono-2-(methacryloyl)ethylsuccinate it is preferred that a further comonomer is present and that this is 2-ethylhexylacrylate. Where the neutral/anionic comonomer is acrylic acid it is preferred that it is used alone with the [2(methacryolyloxy)ethyl]trimethylammonium chloride. More preferably the mol% ratio in the polymerisation mixture of this polymer type is from 50 to 90 [2(methacryolyloxy)ethyl]trimethylammonium chloride the remainder being the non-cationic comonomer(s).

(d) where the cationic comonomer is (ar-vinylbenzyl)trimethylammonium chloride and the neutral/anionic comonomer includes of the anionic comonomers vinylphosphonic acid and/or acrylic acid, and/or of the neutral hydrophilic comonomers N, N-dimethylacrylamide, N-vinylpyrrolidone and/or 2-hydroxyethylacrylate, and/or of the neutral hydrophobic comonomers styrene and/or 2-ethylhexyacrylate. The preferred neutral/anionic comonomer is styrene. Where this comonomer is styrene it is preferred that a second further comonomer is present and that this is either N-[tris(hydroxymethyl)methyl]acrylamide or N-vinylprrolidone. More preferably the mol% ratio in the polymerisation mixture of this polymer type is
from 50 to 90 (ar-vinylbenzyl)trimethylammonium
chloride the remainder being the neutral/anionic comonomer(s)

Preferably the polymer according to the invention is substantially cationic.

The polymer according to the invention is preferably present at from 0.01 to 10% by weight of the composition. Preferably, in an amount ranging from 0.05 to 5% by weight of the composition.

The composition according to the invention may also comprise a halogenated hydroxydiphenyl ether compound, more preferably 2′,4,4′-trichloro-2-hydroxy-diphenyl ether, hereinafter known as triclosan. Preferably the halogenated hydroxydiphenyl ether is present at from 0.01 to 0.5% by weight of the composition. A further preferred group of antimicrobial substances are the parahydroxybenzoic acid esters, also known as parabens, and their structural analogues. Preferred parabens are the medium chain length parabens such as hexyl, heptyl, octyl, nonyl and decyl parabens. Most preferred is the n-octyl paraben.

The composition according to the invention may also comprise a divalent metal salt. Preferably, the divalent metal salt is a salt selected from the group consisting of zinc- and stannous salts such as zinc citrate, zinc sulphate, zinc glycinate, sodium zinc citrate, stannous pyrophosphate and mixtures thereof. The preferable divalent metal salt is zinc citrate.

Suitably, the amount of divalent metal salt ranges from 0.01 to 10% by weight of the composition, preferably from 0.05 to 5% by weight, more preferably from 0.1 to 2% by weight and especially preferably from 0.3 to 0.9% by weight of the composition.

The oral composition according to the invention comprise further ingredients which are common in the art, such as:

antimicrobial agents, e.g. chlorhexidine, sanguinarine extract, metronidazole, quaternary ammonium compounds, such as cetylpyridinium chloride; bis-guanides, such as chlorhexidine digluconate, hexetidine, octenidine, alexidine; and halogenated bisphenolic compounds, such as 2, 2′methylenebis-(4-chloro-6-bromophenol);

anti-inflammatory agents such as ibuprofen, flurbiprofen, aspirin, indomethacin etc.;

anti-caries agents such as sodium- and stannous fluoride, aminefluorides, sodium monofluorophosphate, sodium trimeta phosphate and casein;

plaque buffers such as urea, calcium lactate, calcium glycerophosphate and strontium polyacrylates;

vitamins such as Vitamins A, C and E;

plant extracts;

desensitising agents, e.g. potassium citrate, potassium chloride, potassium tartrate, potassium bicarbonate, potassium oxalate, potassium nitrate and strontium salts;

anti-calculus agents, e.g. alkali-metal pyrophosphates, hypophosphite-containing polymers, organic phosphonates and phosphocitrates etc.;

biomolecules, e.g. bacteriocins, antibodies, enzymes, etc.;

flavours, e.g. peppermint and spearmint oils;

proteinaceous materials such as collagen;

preservatives;

opacifying agents;

colouring agents;

pH-adjusting agents;

sweetening agents;

pharmaceutically acceptable carriers, e.g. starch, sucrose, water or water/alcohol systems etc.;

surfactants, such as anionic, nonionic, cationic and zwitterionic or amphoteric surfactants;

particulate abrasive materials such as silicas, aluminas, calcium carbonates, dicalciumphosphates, calcium pyrophosphates, hydroxyapatites, trimetaphosphates, insoluble hexametaphosphates and so on, including agglomerated particulate abrasive materials, usually in amounts between 3 and 60% by weight of the oral care composition. Preferred abrasives are chalk and silica, more preferably fine ground natural chalk.

Humectants such as glycerol, sorbitol, propyleneglycol, xylitol, lactitol etc.;

binders and thickeners such as sodium carboxymethyl-cellulose, hydroxyethyl cellulose (Natrosol®), xanthan gum, gum arabic etc. as well as synthetic polymers such as polyacrylates and carboxyvinyl polymers such as Carbopol®;

polymeric compounds which can enhance the delivery of active ingredients such as antimicrobial agents can also be included;

buffers and salts to buffer the pH and ionic strength of the oral care composition; and

other optional ingredients that may be included are e.g. bleaching agents such as peroxy compounds e.g. potassium peroxydiphosphate, effervescing systems such as sodium bicarbonate/citric acid systems, colour change systems, and so on.

Liposomes may also be used to improve delivery or stability of active ingredients.

The oral compositions may be in any form common in the art, e.g. toothpaste, gel, mousse, aerosol, gum, lozenge, powder, cream, etc. and may also be formulated into systems for use in dual-compartment type dispensers.

The polymer according to the invention is capable of delivering itself to the oral surfaces during brushing. Preferably, in conjunction with a benefit agent selected from any of those included herein. Most preferable of these benefit agents are the antimicrobials, anti-caries agents, anti-tartar agents, anti-malodour agents and bleaching or tooth whitening agents.

In a second aspect the present invention provides a process for preparing an oral care composition according to any one of claims 1 to 5, comprising the steps of:

preparing a mixture of comonomers as defined in the first aspect of the invention in an ethanol/water diluent;

polymerising the mixture by heating it under inert gas in the presence of an initiator;

extracting the polymer so obtained and blending it with one or more oral care actives and/or excipients so as to produce an oral care composition which is in the form of any one of a toothpaste, gel, foam, chewing gum, deformable

strip or mouthwash and which is suitable for use in the oral cavity.

Preferably, the monomers are mixed at about 20% by (w/v) in ethanol: water mixture of from 50:50 to 95:5, more preferably from 70:30 to 90:10 and most preferably 80:20.

Preferably, the initiator is AIBN and is added at from 0.1 to 5%, preferably from 0.5 to 2.0% and most preferably at 1.0% mol with respect to the total monmomers.

Preferably, the inert gas is argon.

Preferably, the heating step involves heating for up to 36, preferably up to 24 and most preferably for 18 hours at above 45° C., prefereably more than 50° C. and most preferably at aboput 65° C.

The monomer mixture is then preferably cooled to room temperature.

The polymer is then preferably, diluted with ethanol: water of from 50:50 to 95:5, more preferably from 70:30 to 90:10 and most preferably 80:20 to bring the final concentration to about 10% (w/v).

Preferably the reaction is carried out in a well of a 96-well plate.

### EXAMPLES

Manufacture of Polymers

Manufacture of polymers is done by preparing a mixture of comonomers as defined in any one of claims 1 to 3 in an ethanol/water diluent and polymerising the mixture by heating it under inert gas in the presence of an initiator

Assessment of delivery to oral surfaces:

The following polymers were used as control polymers throughout the examples:
1) Pluronic polymer F 127, a polyethyleneoxide-b-polypropyleneoxide-b-polyethylenoxide triblock copolymer having a total molecular weight (M_{w}) of about 12,600 and containing about 70 wt.% polyethyleneoxide units;
2) Gantrez polymer AN-119, a PMA-VE copolymer having a
molecular weight (Mₙ) of about 80,000; and
3) C6 and C12 Gantrez derivatives made by the applicant. The Gantrez polymer described above (#2) was reacted with hexylamine and dodecylamine.

### Example 1

The control polymers were labeled by fluorescein and dissolved in deionized water under stirring to make up stock solutions having a polymer concentration of 80 g/l.

These stock solutions were then diluted (dilution ratio: 40:1) with an artificial saliva composition in order to prepare control polymer formulations in saliva having a polymer concentration of 2 g/l, followed by filtration. The artificial saliva composition was made up according to the method described in Wong, L and Sissons, C H; Archives of Oral Biology 46 (2001 477-486, A comparison of human dental plaque microcosm biofilms grown in an undefined medium and a chemically defined artificial saliva.

Moreover, an artificial saliva composition containing the free dye was prepared.

Additional formulations containing SDS (sodium dodecyl sulfate) were prepared in order to study the effect of a surfactant.

Pig tongue was selected as a control model substrate for soft oral tissue, representing human tongue, gums, etc. The model substrate was pre-treated with a saliva composition overnight. The pre-treated substrate was spotted by the control polymer formulations (500 μl per spot), followed by washing out non-adsorbed polymer by saliva. The pre-treated substrate was also spotted by the saliva formulation containing the free dye.

HAP powder (porous HAP particles having a size of about 20 μm) and HAP discs (discs size: 0.5 inch DIA×0.03 inch×0.05 inch) were selected as model substrates for hard oral tissue, representing the enamel of the human teeth. 50 mg of HAP was put into 800 μl vials (0.45 μm PP filter, UNIFILTER from Whatman). Next, 600 μl of saliva was added to each vial and the HAP suspension was shaked/stirred at least three hours, followed by filtering and drying by air. The substrate was then exposed to the polymer formulations, followed by washing out non-adsorbed polymer by saliva. The substrate was also exposed to the saliva formulation containing the free dye.

The control polymer formulations as well as the artificial saliva formulations containing the free dye were screened for adsorption on both soft and hard oral tissues by using a fluorescence imaging system.

### Example 2

This example demonstrates the screening of polymers for adsorptivity to both hard and soft oral tissues.

The relevant monomers were used for the preparation of polymers. Various homopolymers and copolymers obtained by polymerizing the monomers were labeled by fluorescein and dissolved in deionized water under stirring to make up stock solutions having a polymer concentration of 80 g/l. These stock solutions were diluted (dilution ratio: 40:1) with an artificial saliva composition in order to prepare polymer formulations in saliva having a polymer concentration of 2 g/l, followed by filtration.

Soft and hard oral tissues (pig tongue and HAP powder/discs) were exposed to the polymer formulations in the same manner as in Example 1, and the obtained polymers were screened for adsorption on both soft and hard oral tissues as in Example 1, always accompanied by a control polymer (Pluronic polymer) in order to normalize the response.

TABLE

| | Chemistry | | | | | | | |
|---|---|---|---|---|---|---|---|---|
| | Monomer 1 | | Monomer 2 | | Monomer 3 | | | |
| | Name | % | Name | % | Name | % | A | B |
| 1 | VBTMAC | 60 | Sty | 20 | THMMAM | 20 | 4.3 | 7.8 |
| 2 | VBTMAC | 60 | Sty | 20 | VPL | 20 | 6.0 | 5.8 |
| 3 | DMAPMAM | 60 | MAES | 20 | THMMAM | 20 | 5.2 | 5.0 |
| 4 | DMAPMAM | 60 | MAES | 20 | BA | 20 | 4.8 | 5.4 |
| 5 | DMAPMAM | 90 | VA | 10 | | | 4.1 | 6.1 |
| 6 | VBTMAC | 25 | DMA | 75 | | | 3.1 | 7.1 |
| 7 | VBTMAC | 60 | EHA | 20 | HEA | 20 | 4.6 | 5.1 |
| 8 | MAETMAC | 60 | MAES | 20 | EHA | 20 | 6.3 | 3.3 |
| 9 | DMAPMAM | 75 | DMA | 25 | | | 5.2 | 4.3 |
| 10 | DMAPMAM | 60 | EHA | 20 | PEGMA | 20 | 4.6 | 4.5 |
| 11 | VBTMAC | 50 | VPA | 50 | | | 3.0 | 6.9 |
| 12 | MAETMAC | 90 | AA | 10 | | | 4.2 | 4.7 |
| 13 | DMAPMAM | 33 | VA | 33 | VPL | 33 | 2.7 | 6.8 |
| 14 | DMAPMAM | 60 | AMMPSA | 20 | PEGMEMA | 20 | 4.8 | 3.8 |
| 15 | VBTMAC | 60 | AA | 20 | EHA | 20 | 3.4 | 3.2 |
| 16 | DMAPMAM | 10 | THMMAM | 90 | | | 2.2 | 3.8 |
| 17 | AEMAH | 75 | DMA | 25 | | | 2.4 | 3.0 |
| 18 | DMAPMAM | 60 | AA | 20 | VPL | 20 | 2.3 | 3.5 |
| 19 | MAETMAC | 20 | VPA | 60 | HEA | 20 | 0.2 | 4.4 |
| 20 | VPA | 75 | THMMAM | 25 | | | 0.4 | 4.1 |
| 21 | VPA | 50 | THMMAM | 50 | | | 0.1 | 2.4 |
| 22 | MAETMAC | 20 | MAES | 60 | Sty | 20 | 0.1 | 2.3 |
| 23 | VPA | 50 | VPL | 50 | | | 0.5 | 2.6 |
| 24 | VBTMAC | 50 | MAES | 50 | | | 0.4 | 2.0 |
| 25 | VPA | 75 | DMA | 25 | | | 0.6 | 2.2 |
| 26 | HEA | 100 | | | | | 0.0 | 0.0 |
| 27 | DMA | 100 | | | | | 0.0 | 0.1 |
| 28 | THMMAM | 100 | | | | | 0.0 | 0.1 |
| 29 | VPL | 100 | | | | | 0.0 | 0.0 |
| 30 | DMAEA | 0 | THMMAM | 100 | | | 0.0 | 0.5 |
| 31 | DMAEA | 0 | HEA | 100 | | | 0.0 | 0.5 |
| 32 | VA | 10 | DMA | 90 | | | 0.0 | 0.5 |
| 33 | VA | 10 | THMMAM | 90 | | | 0.0 | 0.5 |

VBTMAC is (ar-vinylbenzyl) trimethylammonium chloride

DMAPMAM is (dimethylaminopropyl) methacrylamide

MAETMAC is [2(methacryloyloxy)ethyl]trimethylammonium chloride

AEMAH is 2-aminoethylmethacrylate hydrochloride

STY is styrene

MAES is mono-2-(methacryloyl)ethyl succinate

VA is vinyl acetate

DMA is N, N-dimethylacrylamide

EHA is 2-ethylhexylacrylate

VPA is vinylphosphonic acid

AA is acrylic acid

AMMPSA is 2-acrylamido-2-methyl-1-propanesulfonic acid

THMMAM is N-[tris(hydroxymethyl)methyl] acrylamide

VPL is-N-vinylpyrrolidone

BA is butyl acrylate

HEA is 2-hydroxyethylacrylate

PEGMEMA is polyethyleneglycol methylethermethacrylate

1. Oral care composition comprising a polymer obtainable by copolymerising a mixture of comonomers, said mixture comprising:
(a) a cationic monomer selected from (ar-vinylbenzyl) trimethylammonium chloride, (dimethylaminopropyl) methacrylamide, [2(methacryloyloxy)ethyl]trimethylammonium chloride, 2-aminoethylmethacrylate hydrochloride and mixtures thereof; and
(b) at least one anionic or neutral monomer selected from styrene, mono-2-(methacryloyl)ethyl succinate, vinyl acetate, N,N-dimethylacrylamide, 2-ethylhexylacrylate, vinylphosphonic acid, acrylic acid, 2-acrylamido-2-methyl-1-propanesulfonic acid, N-[tris(hydroxymethyl)methyl] acrylamide, N-vinylpyrrolidone, butyl acrylate, 2-hydroxyethylacrylate, polyethyleneglycol methylethermethacrylate and mixtures thereof,
said oral care composition in the form of any one of a toothpaste, gel, foam, chewing gum, deformable strip or mouthwash and which is suitable for use in the oral cavity.

2. Oral care composition according to claim 1, comprising a polymer obtainable by copolymerising a mixture of (ar-vinylbenzyl)trimethylammonium chloride, styrene and a further neutral comonomer selected from N-[tris(hydroxymethyl)methyl] acrylamide and N-vinylpyrrolidone.

3. Oral care composition according to claim 1 , comprising a polymer obtainable by copolymerising a mixture of (dimethylaminopropyl) methacrylamide with anionic and/or neutral comonomers selected from mono-2-(methacryloyl)ethyl succinate, vinyl acetate, butyl acrylate, N-[tris(hydroxymethyl] acrylamide and mixtures thereof.

4. Process for preparing an oral care composition according to claim 1, comprising the steps of:
preparing a mixture of comonomers in an ethanol/water diluent; polymerising the mixture by heating it under inert gas in the presence of an initiator;
extracting the polymer so obtained and blending it with one or more oral care actives and/or excipients so as to produce an oral care composition which is in the form of any one of a toothpaste, gel, foam, chewing gum, deformable strip or mouthwash and which is suitable for use in the oral cavity.
